(19) United States
(12) Patent Application Publication
(10) Pub. No.: US 2010/0154794 A1
(43) Pub. Date: Jun. 24, 2010

(54) Inhaler Flow Channel

(76) Inventor: Boris N. Valentin, Struer (DK)

Correspondence Address: JAMES C. WRAY, 1493 CHAIN BRIDGE ROAD, SUITE 300, MCLEAN, VA, 22101, US

(21) Appl. No.: 12/225,352

(22) PCT Filed: Mar. 21, 2007
(86) PCT No.: PCT/DK2007/000140

§ 371 Date: Oct. 27, 2008

Related U.S. Application Data
(60) Provisional application No. 60/784,250, filed on Mar. 21, 2006.

(30) Foreign Application Priority Data
Mar. 21, 2006 (DK) PA 2006 00402

(51) Int. Cl. A61M 15/00

(52) U.S. Cl. 128/203.15; 128/203.12; 128/203.21

(57) ABSTRACT

The present invention provides an inhaler, which separates the drug aerosolization process and the drug delivery process within the inhaler, and controls the inhalation air flow profile in a way, such that transportation of the drug in the inhaler flow channel is enveloped in a laminar air stream presented to the air tract of the user, thereby eliminating drug deposition on the inhaler inside walls. The inhaler invention therefore provides advantages over other inhalers, especially, when a repeatable emitted dose is important and generally, where contamination and hygiene in the flow channel is an issue as is the case with multi-dose inhalers.

### FIELD OF THE INVENTION

The present invention relates generally to the field of pulmonary delivery of pharmaceuticals and drugs. Particular use of the present invention is found in delivery of metered and packaged dry powder medications and drugs for inhalation therapy and will be described in connection with such use, although other uses are contemplated, including liquid medication applications.

Certain diseases of the respiratory tract are known to respond to treatment by the direct application of therapeutic agents. As these agents are most readily available in dry powdered form, their application is most conveniently accomplished by inhaling the powdered material through the nose or mouth. This powdered form results in the better utilization of the medication in that the drug is deposited exactly at the site desired and where its action may be required; hence, very minute doses of the drug are often equally as efficacious as larger doses administered by other means, with a consequent marked reduction in the incidence of undesired side effects and medication cost. Alternatively, the drug in powdered form may be used for treatment of diseases other than those of the respiratory system. When the drug is deposited on the very large surface areas of the lungs, it may be very rapidly absorbed into the blood stream; hence, this method of application may take the place of administration by injection, tablet, or other conventional means.

It is the opinion of the pharmaceutical industry that the bioavailability of the drug is optimum when the drug particles delivered to the respiratory tract are between 1 to 5 microns in size. When the drug particles need to be in this size range the dry powder delivery system needs to address a number of issues:

(1) Small size particles develop an electrostatic charge on themselves during manufacturing and storage. This causes the particles to agglomerate or aggregate, resulting in clusters of particles which have an effective size greater than 5 microns. The probability of these large clusters malting it to the deep lungs then decreases. This in turn results in a lower percentage of the drug being available to the patient for absorption.

(2) The amount of active drug that needs to be delivered to the patient may be of the order of tens of micrograms. Since current powder filling equipment cannot effectively deliver aliquots of drugs in microgram quantities with acceptable accuracy, the standard practice is to mix the active drug with a filling or bulking agent such as lactose. This additive also makes the drug “easy to flow”. In some cases this filler is sometimes called a carrier. These carrier particles are often larger than the drug particles in size. The ability of the dry powder inhaler to separate drug from the carrier is an important performance parameter in the effectiveness of the design.

(3) Active drug particles with sizes greater than 5 microns will be deposited either in the mouth or throat. This introduces another level of uncertainty since the bioavailability and absorption of the drug in these locations is different from the lungs.

Dry powder inhalers need to minimize the drug deposited in these locations to reduce the uncertainty associated with the bioavailability of the drug.

Prior art dry powder inhalers (DPIs) usually have a means for introducing the drug (active drug plus carrier) into a high velocity air stream. The high velocity air-stream is used as the primary mechanism for breaking up the cluster of micronized particles or separating the drug particles from the carrier. Several inhalation devices useful for dispensing this powder form of medication are known in the prior art. For example, in U.S. Pat. Nos. 3,507,277; 3,518,992; 3,635,219; 3,795,244; and 3,807,400, inhalation devices are disclosed having means for piercing or removing the top of a capsule containing a powdered medication, which upon inhalation is drawn out of the pierced or topped capsule and into the user's mouth. Several of these patents disclose propeller means, which upon inhalation aid in dispensing the powder out of the capsule, so that it is not necessary to rely solely on the inhaled air to suction powder from the capsule. For example, in U.S. Pat. No. 2,517,482, a device is disclosed having a powder containing capsule placed in a lower chamber before inhalation, where it is pierced by manual depression of a piercing pin by the user. After piercing, inhalation is begun and the capsule is drawn into an upper chamber of the device where it moves about in all directions to cause a dispensing of powder through the pierced hole and into the inhaled air stream. U.S. Pat. No. 3,831,606 discloses an inhalation device having multiple piercing pins, propeller means, and a self-contained power source for operating the propeller means via external manual manipulation, so that upon inhalation the propeller means aids in dispensing the powder into the stream of inhaled air. See also U.S. Pat. No. 5,458,135.

Another type of inhalers uses liquid based drugs when administering to the patient.

An example of such liquid dispensing device is disclosed in US 2003/0072717 describing a device comprising a reservoir for storing the compound where said reservoir is fluidly connected to a system which system generates liquid particles and emits these. The reservoir as well as the system for emitting the particles is placed in a housing. One end of the housing is suitable to be used as a mouthpiece such that a patient when desiring to get a dose inserts the housing in the mouth and sucks air through the housing so that an air flow is created inside the housing around the reservoir and device for emitting the particles. Although the description declares that a substantially non-turbulent and laminar air flow as well as an unobstructed air flow is created inside the housing, this is for physical reasons not entirely true. The device for delivering the particles is arranged in the middle of the housing and therefore obstructs part of the air flow through the housing, whereby differences in air speed will occur over a cross section of the air flow. Along the interior sides of the housing the air flow will be faster than the air flow immediately downstream of the device to emit the droplets placed in the housing. This gives rise to turbulence and an uneven distribution of the droplets emitted by the device arranged inside the housing such that the medication will be unevenly distributed across the cross section of the housing and thereby the delivered dose to the patient may not always be as should be predicted. It is evident that the arrangement of the system for emitting the droplets inside the housing and thereby in the air flow created by the patient sucking air through the device will influence the air flow and will create turbulence such that a laminar air flow will not be achieved over the entire cross section. Furthermore, the device for emitting the droplets which according to the description is comparable to an inkjet system requires a source of energy as well as a special type of compounds in that the emitting device may clog up or dry out so that further dispensation of compound doses is prevented due to the system being clogged up.

From U.S. Pat. No. 5,894,841 is also known a device where a compound in liquid shape is dispensed by means of e.g. a bubble jet or piezo electric emitting device and where an air stream is created inside a housing due to a patient sucking on a mouthpiece of the device, whereby an air stream is supposed to surround the emitted droplets and thereby guide them into the mouth of the user. The device comprises a number of fragile components and at the same time the air stream will not be able for the same reasons as explained above with reference to US2003/0072717 to create a laminar air flow through the device and thereby ensuring a constant air flow across the section of the mouthpiece, whereby it may be ensured that the dose which the patient was expecting, is actually delivered.

These prior art devices present several problems and possess several disadvantages. For instance, these prior art devices require that the user exert considerable effort in inhalation to effect dispensing or withdrawal of powder from a pierced capsule into the inhaled air stream. With these prior art devices, suction of powder through the pierced holes in the capsule caused by inhalation generally does not withdraw all or even most of the powder out of the capsule, thus causing a waste of the medication. Also, such prior art devices may result in uncontrolled amounts or clumps of powdered material being inhaled into the user's mouth, rather than a constant inhalation of controlled amounts of finely dispersed powder.

The above description of the prior art is taken largely from U.S. Pat. No. 3,948,264 to Wilke et al, who disclose a device for facilitating inhalation of a powdered medication that includes a body portion having primary and secondary air inlet channels and an outlet channel. The secondary inlet channel provides an enclosure for a capsule containing the powdered medication, and the outlet channel is formed as a mouthpiece protruding from the body. A capsule piercing structure is provided, which upon activation forms one or more holes in the capsule so that upon vibration of the capsule by an electro-mechanical vibrator, the powdered drug may be released from the capsule.

The piercing means disclosed in Wilke et al includes three radially mounted, spring-biased piercing needles mounted in a trochoidal chamber. Upon hand rotation of the chamber, simultaneous inward radial motion of the needles pierces the capsule. Further rotation of the chamber allows the needles to be retracted by their spring mountings to their original positions to withdraw the needles from the capsule. The electro-mechanical vibrator includes, at its innermost end, a vibrating plunger rod, which projects into the intersection of the inlet channel and the outlet channel. Connected to the plunger rod is a mechanical solenoid buzzer for energizing the rod to vibrate. The buzzer is powered by a high energy electric cell and is activated by an external button switch.

According to Wilke et al, upon inhalation through outlet channel and concurrent pressing of switch to activate the electromechanical vibrating means, air is sucked through the inlet channels and the air stream through the secondary inlet channel raises the capsule up against the vibrating plunger. The capsule is thus vibrated rapidly with powder being fluidized and dispensed from the pierced holes therein. The air stream through inlet channel aids in withdrawal of powder from the capsule and carries this powder through the outlet channel to the mouth of the user. Wilke et al. further discloses that the electromechanical vibrator means may be placed at a right angle to the inlet chamber and that the amplitude and frequency of vibration may be altered to regulate dispensing characteristics of the inhaler.

The prior art devices have a number of disadvantages which makes them less than desirable for the delivery of dry powder to the lungs. Some of these disadvantages are:
The performance of the prior art inhalers depends on the flow rate generated by the user. Lower flow rate does not result in the powder being totally de-agglomerated and hence adversely affects the dose delivered to the patient.
Inconsistency in the bioavailability of the drugs from dose-to-dose because of lack of consistency in the de-aggregation process.
Inconsistency in the bioavailability of the drugs from dose-to- dose because of drug dose partially depositing on the inside wall of the inhaler flow channel.
Large energy requirements for driving the electromechanical based inhalers which increases the size of the devices making them unsuitable for portable use.
Loss of medication from opened or topped capsules.
Deterioration of medication in open or topped capsules due to exposure to oxygen or moisture.

In prior U.S. Pat. Nos. 6,026,809 and 6,142,146, Gumaste provides an inhaler that utilizes a vibrator to facilitate suspension of a medication or drug into a gas that overcomes the aforesaid and other disadvantages and drawbacks of the above prior art. More particularly, the inhaler of the aforesaid patent includes a piezoelectric vibrator for de-aggregating the medication or drug and driving the de-agglomerated medication or drug into suspension. US Pat. appl. No. 2005/0183724 to Gumaste discloses a refined method of dose release implementing the synthetic jet principle into the device.

However, the aforementioned prior art devices does not disclose effective means for solving the problem, inherent with most inhalers, that a certain amount of the aerosolized drug will deposit on the walls of the flow channel and mouthpiece. The amount of drug deposited depends on the patient's inhalation flow profile, electrostatic properties of the drug and moisture deriving from a patient accidentally exhaling into the device. The deposited layer of drug will increase with use and may impose serious effective dose control problems over time and furthermore represents a hygienic risk to the patient.

### DESCRIPTION OF THE INVENTION

The present invention provides an inhaler design, in which the drug aerosolization process and the drug delivery process are separated in the inhaler, and controls the inhalation air flow profile in a way, that transportation of the drug in the inhaler flow channel is enveloped in a laminar air stream presented to the air tract of the user, thereby eliminating drug deposition on the inhaler inside walls. The invention therefore provides advantages over previous inhalers, especially, when a repeatable emitted dose is important and generally, where contamination and hygiene is an issue with multi-dose inhalers.

More particularly, the invention discloses a flow channel design, where the flow channel shape is essentially a tube, which is closed in one end by a wall and forms the inner wall of a mouthpiece in the other end. In the center of the end wall there is a protrusion into the flow channel. In the center of the protrusion, one or more holes enables aerosolized drug to pass into the flow channel. Close to the end wall an evenly distributed air inlet enables air to flow radially towards the aerosol exit holes. By careful fluid design of the protrusion, air inlets and air distribution to the air inlets, the aerosol will be enveloped in the air stream and transported to the air tract of the user without depositing on the walls of the flow channel.

The invention works over the entire inhalation flow rate span usually associated with inhalation therapies, but the invention also considers air restrictors to be adjusted to optimize drug deposition in the lungs for a specific drug.

Applications include powdered and liquid based drugs.

Drug packaging principles include, but are not limited to blisters, capsules, canisters, bulk powder and multiple liquid dose bags.

Drug aerosolization principles include, but are not limited to ultrasound, electromechanical shakers, nozzles, compressed air, heating and combinations of such principles.

Therapeutic areas include, but are not limited to respiratory diseases, diabetes, allergy and pain killing.

For those skilled in the art it is obvious that this invention may be combined with dose counters, cap blocking of dose release, return blocking means to avoid over-counting of doses, breath activated dose release, drug coding elements and patient compliance feedback i. e. as for example described in prior art WO 04/041334, US 2005/0087473, and WO 01/703115.

### DESCRIPTION OF THE DRAWING

FIG. 1 shows a cross cut of an embodiment of a flow channel,

FIG. 2 shows an embodiment of a flow channel with a mouthpiece,

FIG. 3 shows an embodiment of an exterior manifold design,

FIG. 4 shows a cross cut of a manifold air distributors with restrictors,

FIG. 5 shows a flow channel with a blister,

FIG. 6 shows a flow channel to be mounted with nozzle and a canister,

FIG. 7 shows some variations of intrusion geometries, and

FIG. 8 shows a flow channel simulation for an embodiment with a blister

### DETAILED DESCRIPTION OF THE INVENTION

Most commonly, powdered drugs for pulmonary administration are either packed as metered doses of agglomerated powder in laminated plastic and alumina foil capsules or blisters, which are pierced and shaken to bring the powder into the inhalation flow channel of the inhaler, or alternatively, the powder is supplied in a bulk reservoir in the inhaler and metered before or during an inhalation procedure.

The purpose of the flow channel is to de-agglomerate the powder into fine aerosol particles with a preferred particle size of 1-5 micrometer by introducing turbulent flow in the flow channel and thereafter to transport and deliver the aerosol to the inhaler mouthpiece and into the user's airways by the inhalation flow.

Drugs supplies in liquid form are usually distributed in a pressurized container called a canister. The canister employs a dose metering valve and upon release of a dose the liquid is aerosolized through a nozzle into the flow channel, where the aerosol droplets are further vaporized and transported to the inhaler mouthpiece and delivered to the user's airways by the inhalation flow.

Many inhalers on the marked, especially those based on a powder formulated drugs, depend on a turbulent flow in the pathway from point of drug release to the mouthpiece of the inhaler, in order to, more or less successfully, de-agglomerate the powder particles into a preferred particle size of 1-5 micrometer. However, the turbulent flow in this flow channel has the drawback that drug particles come into contact with the walls and mouthpiece of the inhaler and some of the particles will adhere and deposit on the surfaces, an effect, that may accelerate over time. The degree of deposition depends on the user's inhalation flow profile, static electric properties of the drug particles and presence of moisture from breathing and surroundings.

The consequences are obvious; primarily the effective dose presented to the user's air tract will vary through the lifetime of the device, which has become an increasing problem, now that inhalers move into dose critical therapies like diabetes; secondly the deposited material is subject to bacterial contamination and therefore presents a hygienic risk to the user.

This invention discloses a generic way to solve such internal deposition problems within inhalers:

The first step is to separate the aerosolization process from the aerosol transporting process. Such aerosolization techniques are well known from prior art i. e. mechanical or ultrasonic vibration of pierced powder capsules and blisters, ultrasonic nebulizing of liquid drug droplets, heating of liquid drugs, canisters with spraying nozzles, establishing air stream through multiple holes in blisters and capsules from a source of pressurized gas, and combinations of these techniques.

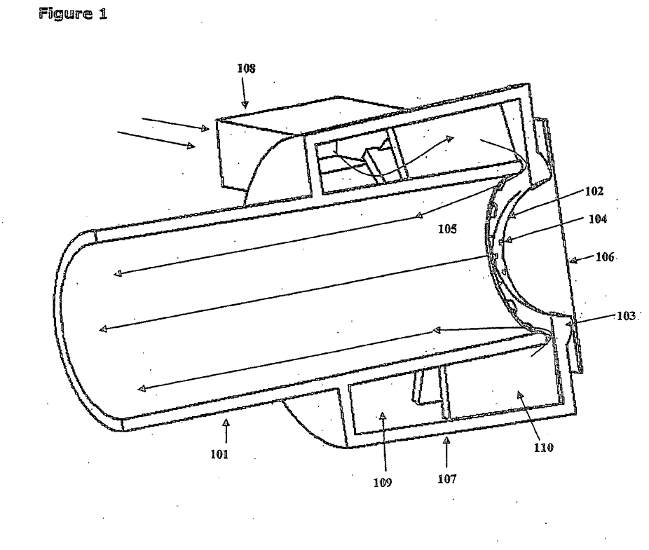

Secondly, a flow channel embodiment FIG. 1, essentially cylindrically shaped 101, essentially closed in one end with a wall constituted by foundation 103 and (in this embodiment) a pierced blister 106 forming a protrusion 102 into the flow channel 101, is attached to the aerosolizing source on the outer side of the end wall 103. The pierced holes 104 enable the aerosolizer to inject a de-agglomerated drug aerosol into the flow channel 101. Close to the end wall, evenly distributed slits or holes 105 in the flow channel enables air to flow radially towards the holes in the protrusion 102. The injected aerosol will be transported, enveloped in a laminar air flow, and delivered to the air tract of the user without having collided with the walls of the flow channel. The conditions for achieving laminar flow (all gas and drug particles have a positive flow component with respect to the intended flow direction and all particles in any cylindrical sheet being concentric with the flow channel having essentially the same velocity) in the flow channel are:

The shape of the protrusion 102 is construed between a large diameter, low height, spherical shape and a high conical shape. All edges and corners in the air inlets 105 and the protrusion 102 should be beveled to avoid local turbulences.

The acceptable inhalation flow rate is constrained between 5 and 100 liters per minute (Includes most practical inhalation flow rates usually recommended between 15 and 60 liters per minute).

The flow channel should form the inner diameter of the mouthpiece. No sudden steps in diameter or shape should be allowed to avoid local turbulences.

The preferred flow channel aspect ratio of length to diameter should be at least 1.

The distributed air inlets 105 should be carefully balanced to avoid skewness of the air stream.

The balanced air flow is provided by a manifold 107 having an inlet port 108, a distribution chamber 109 and axial restrictors 110. Other embodiments are applicable i. e. distributed axial inlets or axial inlet port. In most practical cases one inlet port is preferred to be able to include a breath actuated dose release mechanism to achieve optimal coordination between dose release and user inhalation flow profile. Alternatively any kind of differential pressure sensoring means, measuring i. e. on both sides of the distributed air inlets 105, where the pressure drop is maximal, could be implemented to control dose release timing.

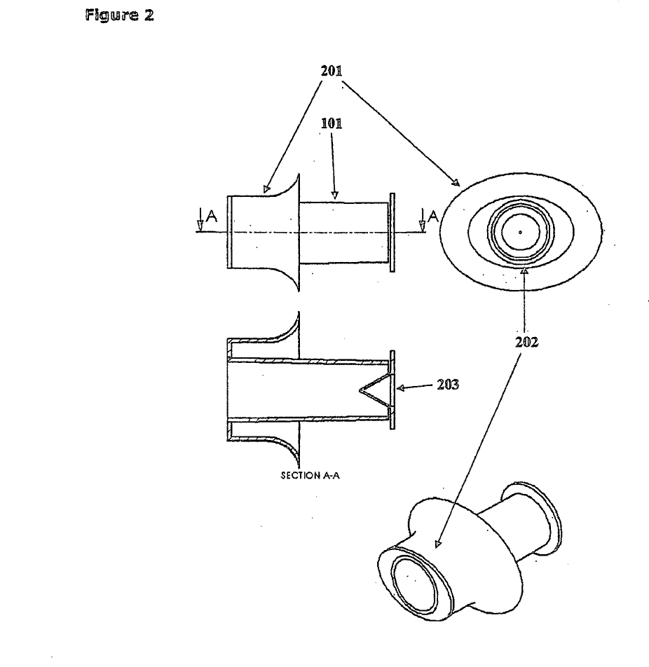

FIG. 2 shows an embodiment of a flow channel 101 with a conically formed blister 203 and an embodiment of a mouthpiece 201, which illustrates that though the inner shape of the mouthpiece is essentially determined by the flow channel 101, virtually any ergonomically convenient outer shape 202 of the mouthpiece may be modeled. The outer shape shown is oval to allow the lips of the user to form an air tight communication between the inhaler and the user's air tract.

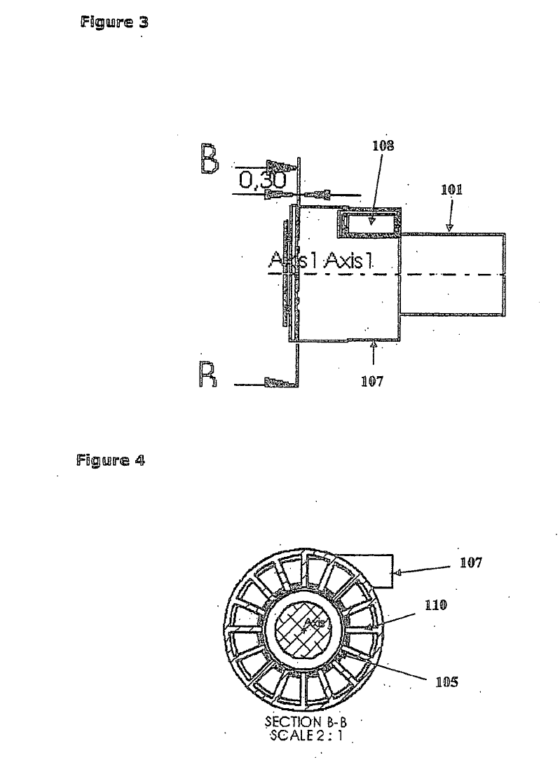

FIGS. 3 and 4 shows an embodiment of a flow channel 101 and the manifold 107 with air distribution means of multiple axial ribs 110 to achieve an even air distribution to the flow channel input slits or holes 105.

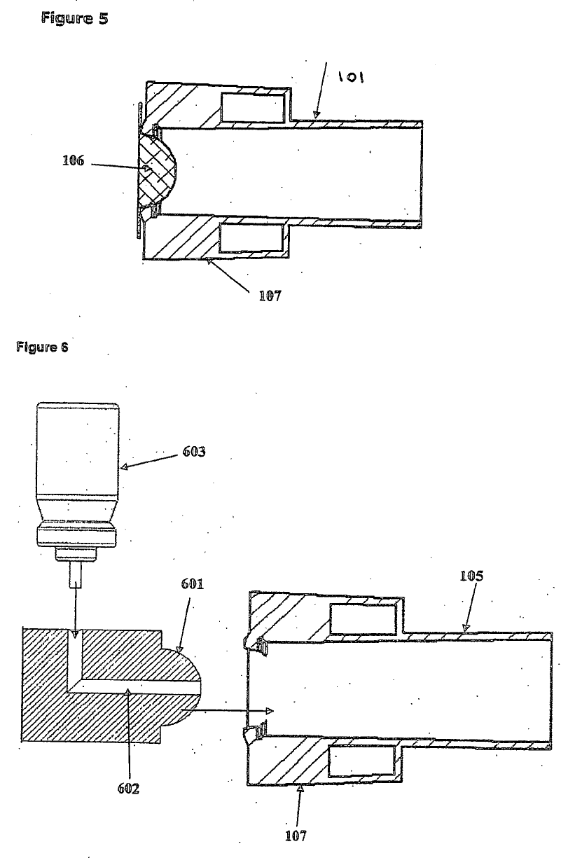

FIG. 5 shows a cross section of the flow channel 101 with manifold 107 loaded with a semi-spherical blister 106.

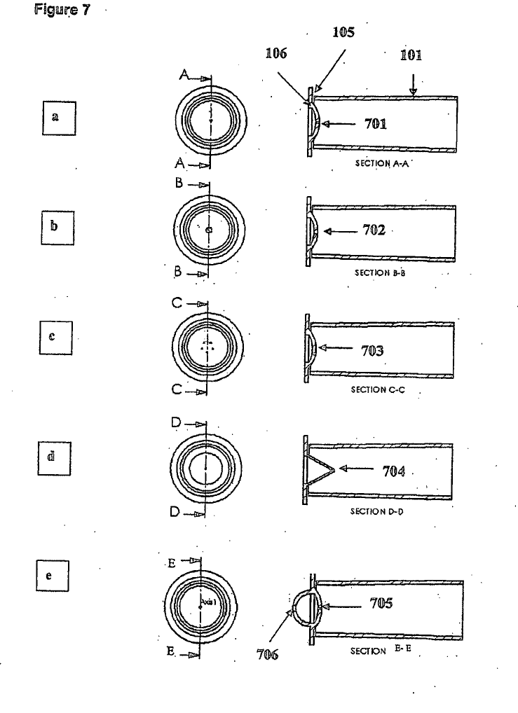

FIG. 6 shows a flow channel 101 with a manifold 107, and a fit for a spherical nozzle 601 with a channel 602 into which a canister 603 can be fitted. This embodiment enables the invention to be used with fluidly formulated pressurized drugs.

FIG. 7, a-e shows a selection of geometries of the protrusion and the aerosol holes. It should be emphasized that parts of the protrusion could be formed by a blister:

a. A single center hole 701 in a large diameter spherical shape.

b. A single center nozzle 702 in a large diameter spherical shape for fluidly formulated drugs.

c. A multi-hole pattern 703 in a large diameter spherical shape.

d. A single center hole 704 in a large beveled cone shape.

e. A single center hole 705 in a large diameter spherical shape. The blister additionally has a negative dome shaped wall with an additional hole 706 for dose release and ejection generated from compressed air or the user's inhalation flow. A pierced elongate powder capsule might also be placed inside the double domed volume for aerosolization.

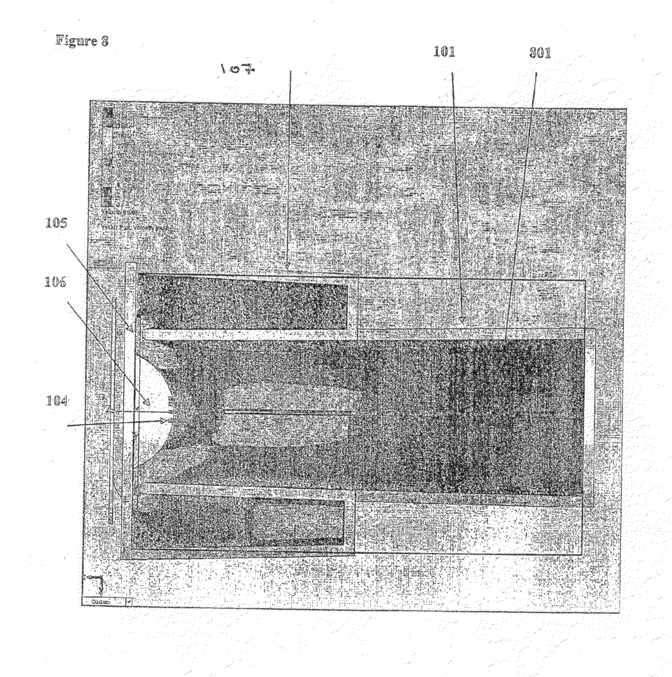

FIG. 8 shows a flow simulation in an embodiment of a flow channel with a single radial air intake manifold and a multi-hole pierced spherically shaped blister. The flow simulation clearly shows that the airflow in the flow channel is laminar after the aerosol injection point and that the aerosol 801 stays close to the center all the way through the flow channel. With the shown geometry, laminar flow conditions exist within an inhalation flow range of 10-90 liters per minute, but adjusting parameters like flow channel aspect ratio, air inlet geometry and balance, and blister dome geometry the optimal flow range may be optimized for a given application.

Various additional changes may be made in the foregoing without departing from the spirit and scope of the protection as afforded by appended claims.

1. Inhaler comprising a drug delivery flow channel and means for connecting said drug delivery flow channel to a drug reservoir, characterized by a first end of said flow channel is suitable to be inserted into the mouth of a user, and that the opposite end of the flow channel is delimited by an end wall and that further in a central part of the end wall a protrusion is provided, said protrusion is in contact with an opening of the drug reservoir, and that the protrusion is provided with one or more holes, and that close to the end wall of the flow channel, air inlets are arranged adjacent the end wall of the flow channel, said air inlets providing a laminar air flow.

2. Inhaler according to claim 1, characterized by the end wall is the cover on one drug dose packaged in a multi-dose blister pack, and that means are provided for piercing the cover on the drug dose.

3. Inhaler according to claim 1, characterized by an inlet manifold is arranged in connection with the air inlets, where said manifold comprises one or more inlet ports, a distribution chamber in which chamber one or more axial restrictors are provided for directing the air flow to the air inlets flow channel.

4. Inhaler according to claim 1, characterized by the air inlets are arranged such that the air flow into the flow channel is in radial direction in relation to the drug carrying air flow.

5. Inhaler according to claim 1, characterized by the air flow is designed to be between 5 liters and 100 liters per minute, more preferred between 15 liters and 60 liters per minute.

6. Inhaler according to claim 1, characterized by the shape of the protrusion in the end wall is selected among: a convex shape, a conical shape, a spherical shape, and that all edges and corners are beveled.

7. Inhaler according to claim 1, characterized by means for introducing the drug into the air flow are provided, and that said means may include mechanical means, ultrasonic means, vibrating means, electromechanical means, nozzle means, compressed air, vaporizing means alone or in any combination.

8. Inhaler according to claim 1, characterized by the drug may be in liquid or powdered form, where the drugs may be packaged in any suitable manner such as blisters, capsules, canisters, bulk powder, single or multiple liquid dose bags.

9. Method for delivering a drug through an inhaler, where the drug is enveloped in a laminar air flow in a drug delivery flow channel, which channel in one end is in communication with a drug supply, and in the opposite end is in communication with a user.
